(19) United States
(12) Patent Application Publication
(10) Pub. No.: US 2017/0086980 A1
(43) Pub. Date: Mar. 30, 2017

(54) ACETABULAR CUP TAPER COVER AND LINER TRIAL

(71) Applicant: DEPUY SYNTHES PRODUCTS, INC., RAYNHAM, MA (US)

(72) Inventor: JOEL ERIC SUCKOW, CHARLOTTE, NC (US)

(21) Appl. No.: 14/864,964

(22) Filed: Sep. 25, 2015

(51) Int. Cl. A61F 2/34

(52) U.S. Cl. CPC A61F 2/34; A61F 2002/3483; A61F 2002/3225

(57) ABSTRACT

An acetabular shell system. The shell system includes an acetabular shell having a convex outer portion and a concave inner portion. The shell system also includes a polymer sleeve having an outer portion sized and shaped to engage at least a portion of the concave inner portion of the acetabular shell.

### FIELD OF THE INVENTION

This present invention is directed to a prosthetic shell assembly, and more specifically, a shell system including an acetabular shell and a protective sleeve.

### BACKGROUND

A joint within the human body forms a juncture between two or more bones or other skeletal parts. The ankle, hip, knee, shoulder, elbow and wrist are just a few examples of the multitude of joints found within the body. As should be apparent from the above list of examples of joints, many of the joints permit relative motion between the bones. For example, the ankle permits a hinge movement, the knee allows for a combination of gliding and hinge movements and the shoulder and hip permit movement through a ball and socket arrangement.

The joints in the body are stressed or can be damaged in a variety of ways. Gradual wear and tear is imposed on the joints through the continuous use of a joint over the years. The joints that permit motion have cartilage positioned between the bones providing lubrication to the motion and also absorbing some of the forces direct for the joint. Over time, the normal use of a joint may wear down the cartilage and bring the moving bones in a direct contact with each other. In contrast, in normal use, a trauma to a joint, such as the delivery of a large force from an automobile accident for example, may cause considerable damage to the bones, the cartilage or to other connective tissue such as tendons or ligaments.

Arthropathy, a term referring to a disease of the joint, is another way in which a joint may become damaged. One form of joint disease is arthritis, which is generally referred to a disease or inflammation of a joint that results in pain, swelling, stiffness, instability, and often deformity.

There are many different forms of arthritis, with osteoarthritis being the most common and resulting from the wear and tear of a cartilage within a joint. Another type of arthropathy is osteonecrosis, which is caused by the death of a part of the bone due to loss of blood supply and subsequent degeneration of the cartilage. Other types of arthritis are caused by trauma to the joint while others, such as rheumatoid arthritis, Lupus, and psoriatic arthritis destroy cartilage and are associated with the inflammation of the joint lining.

The hip joint is one of the joints that is commonly afflicted. The hip joint is a ball and socket joint that joins the femur or thighbone with the pelvis. The pelvis has a hemispherical socket called the acetabulum for receiving the head of the femur. Both the head of the femur and the acetabulum are coated with cartilage for allowing the femur to articulate within the pelvis. Other joints commonly afflicted include those of the spine, knee, shoulder, elbow, carpals, metacarpals, and phalanges of the hand. One means to address this affliction is arthroplasty which commonly refers to the making of an artificial joint. In severe cases of arthritis or other forms of arthropathy, such as when pain is overwhelming or when a joint has a limited range of mobility, a partial or total replacement of the joint may be justified. The procedure for replacing the joint varies, of course, with the particular joint in question, but in general involves replacing a terminal portion of an afflicted bone with a prosthetic implant and inserting a member with structural support to serve as a substitute for the cartilage.

The prosthetic implant is formed of a rigid material that becomes bonded with the bone and provides strength and rigidity to the joint and a bearing member chosen to allow for lubrication to the joint. Suitable materials for the implant include metals and composite materials such as titanium, cobalt chromium, stainless steel, ceramic and suitable materials for the bearing include polyethylene, metal and ceramics. A cement may also be used to secure the prosthetic implant to the host bone.

Total hip replacement, for example, involves removing the ball shaped head of the femur and inserting a stemmed implant into the center of the bone, which is referred to as the medullary canal of the bone. The stem implant may be cemented into the medullary canal or may have a porous coated surface for allowing the bone to heal directly to the implant. The stemmed implant has a neck and a ball shaped head, which are intended to perform the same functions as the neck and head of a healthy femur. The acetabulum of the patient is reamed to receive a shell and liner. A polyethylene, metal, or ceramic liner with a metal shell is inserted into the acetabulum and acts as socket for receiving the head on the stemmed implant. In many current shell and liner constructs, the shell has a female taper adapted to engage a portion of the liner.

Prior to inserting the liner into the shell, it is desirable to protect the taper from any scratching. Also, it would be desirable to have a protector that is also able to function as a trial liner.

### SUMMARY

According to one embodiment of the present invention, an acetabular shell system is provided. The shell system includes an acetabular shell having a convex outer portion and a concave inner portion. The shell system also includes a polymer sleeve having an outer portion sized and shaped to engage at least a portion of the concave inner portion of the acetabular shell.

According to another aspect of this invention, the concave inner portion includes a locking mechanism and the polymer sleeve extends over the locking mechanism.

According to another aspect of this invention, the locking mechanism includes a female taper and the outer portion of the polymer sleeve includes a taper to engage the female taper.

According to another aspect of this invention, the locking mechanism includes a female taper and the outer portion of the polymer sleeve is cylindrical.

According to another aspect of this invention, when the shell and sleeve are assembled, the sleeve including a rim which extends above of the inner portion of the shell.

According to another aspect of this invention, the sleeve includes a pull tab extending beyond the outer portion of the sleeve which engages the shell.

According to another aspect of this invention, the inner portion of the shell includes an apex and the sleeve includes an extension portion that extends toward the apex of the shell.

According to another aspect of this invention, the apex of the shell includes a screw hole and the extension portion of the sleeve extends around the screw hole.

According to another aspect of this invention, wherein the extension portion of the sleeve includes a cap which extends over the apex of the shell.

According to another embodiment of the present invention, a method for performing hip arthroplasty is provided. The method includes inserting an acetabular shell into a prepared acetabulum. The acetabular shell has a taper and a sleeve covering the taper. The head is inserted into the shell and sleeve. The range of motion of the head in the shell and sleeve is tested and the sleeve is then removed from the shell.

### BRIEF DESCRIPTION OF FIGURES

FIG. 1 is a perspective view of an acetabular shell according to one embodiment of the present invention.

FIG. 2 is a perspective view of a sleeve according to one embodiment of the present invention.

FIG. 3 is a perspective view of the sleeve of FIG. 1 inserted into the acetabular shell of FIG. 1.

FIG. 4 is a perspective view of a sleeve and shell assembly according to another embodiment of the present invention.

FIG. 5 is a perspective view of a sleeve and shell assembly according to yet another embodiment of the present invention.

FIG. 6 is a perspective view of a sleeve and shell assembly according to yet another embodiment of the present invention.

FIG. 7 is a view of a trial liner and head of the prior art.

FIG. 8 is a flow chart illustrating the use of a sleeve according to one embodiment of the present invention.

### DETAILED DESCRIPTION

An acetabular shell 10 according to one embodiment of the present invention is shown. As shown, the shell 10 includes a convex outer portion 12 and a concave inner portion 14. The convex outer portion 12 is inserted into a surgically prepared cavity in a patient's acetabulum during surgery. In this embodiment, the convex outer portion 12 includes a coating to enhance bone-ingrowth. In other embodiments, no coating may be included or other types of coatings or materials may be used. The concave inner portion 14 is designed to receive a liner (not shown) which will engage a head on a stem. The liner may be locked into the shell via a locking mechanism 15. In this embodiment, the locking mechanism includes a female taper 16 and a locking ring 18. Anti-rotation devices (ARDs) 20 are also included on the inner portion 14 of the shell.

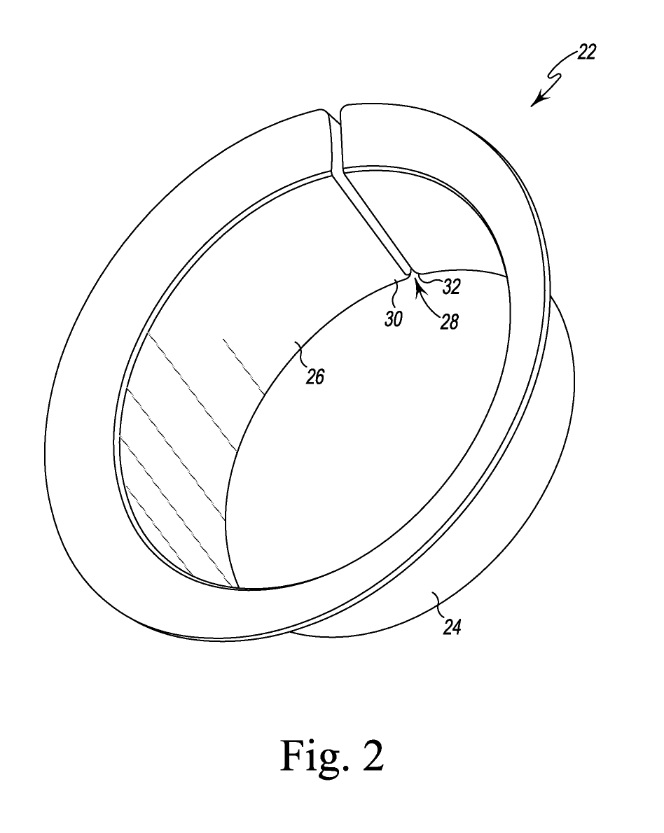

Turning now to FIG. 2, an embodiment of a sleeve 22 according is illustrated. The sleeve is a polymer, such an ultrahigh molecular weight polyethylene, PEEK, or other polymer used in medical devices. The sleeve 22 includes an outer portion 24 and an inner portion 26. In this embodiment, the sleeve 22 is a single piece curved around with a single slit 28 between its two ends 30, 32.

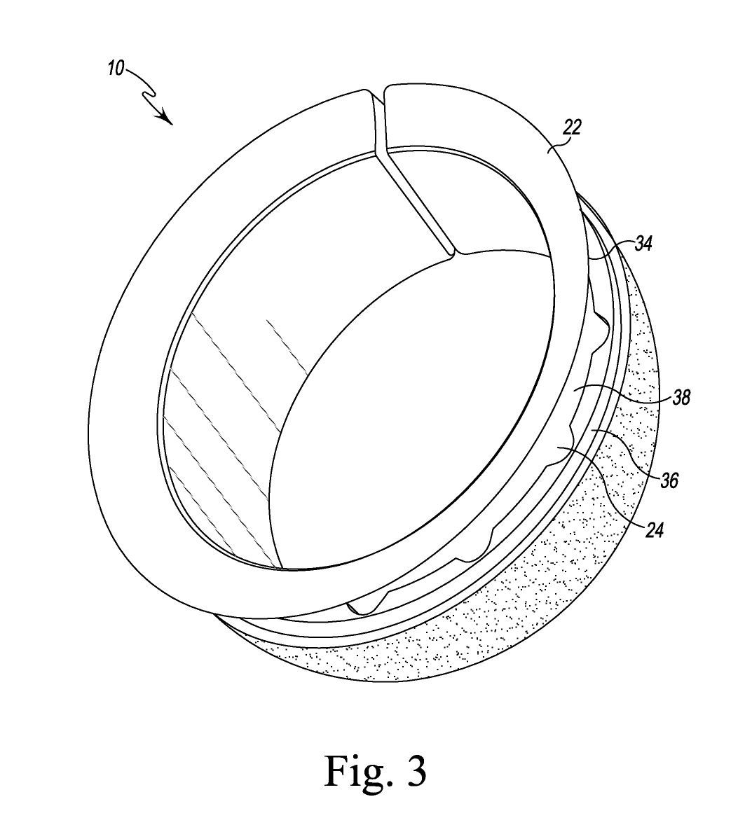

The outer portion 24 engages the inner portion 14 of the shell 10 (FIG. 3) and extends over the locking mechanism 15 of the shell 10. The sleeve 22 protects the female taper 16 from debris and from becoming scratched. In some embodiments, the outer portion 24 of the sleeve 22 is tapered to match the taper of the inner portion 14 of the shell 10. In other embodiments, the outer portion 24 is cylindrical in shape and a portion of the outer portion 24 will rest against the tapered portion of the inner portion 14 of the shell 10. As shown in FIG. 3, when the sleeve 22 is inserted into the shell 10, a rim 34 of the sleeve 22 extends above a rim 36 of the shell 10. The rim 34 and portion 38 of the sleeve 22 that extend above the rim 36 of the shell 10 may allow a user to easily grasp the sleeve 22 in order to remove the sleeve 22 from the shell 10.

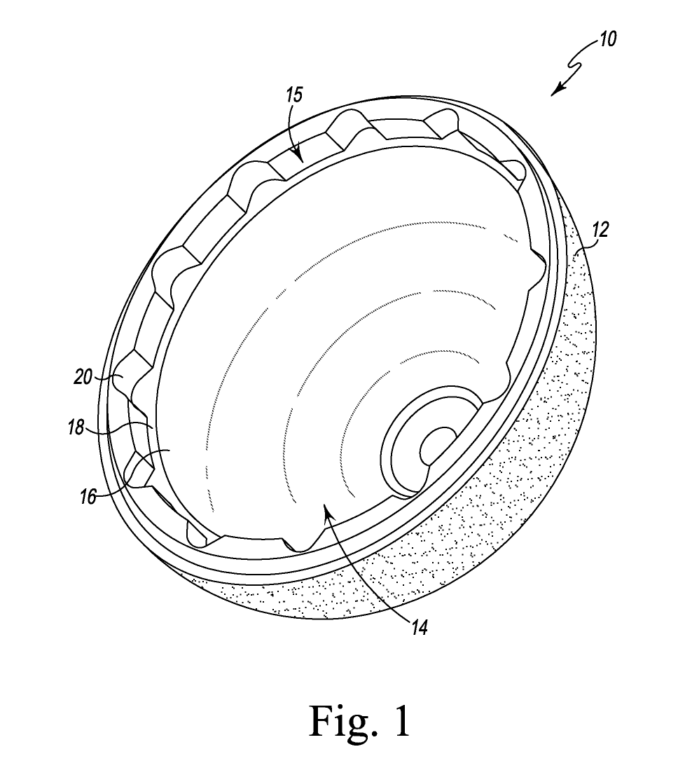

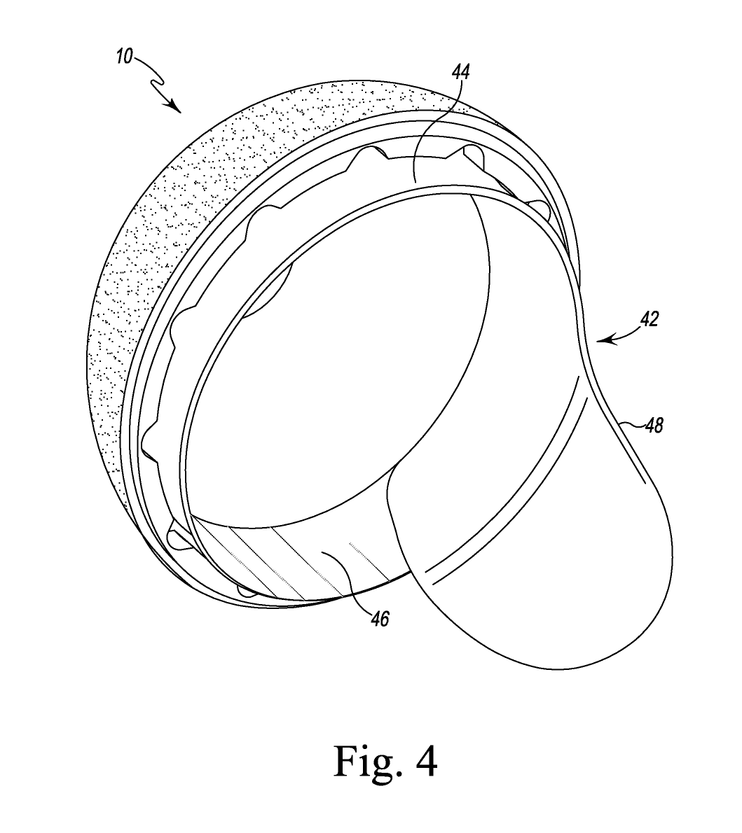

Turning now to FIG. 4, another embodiment of a sleeve 42 is illustrated. In this embodiment, the sleeve 42 includes most of the same features as the sleeve 22 illustrated in FIGS. 1-3. The sleeve 42 includes an outer portion 44 and an inner portion 46. The outer portion 44 engages the inner portion 14 of the shell 10. In this embodiment, the sleeve 42 is a single piece curved around with a pull tab 48 at one end. The pull tab 48 allows a user to easily grasp the sleeve 42 and remove it from the shell 10. As in the embodiment shown in FIGS. 1-3, the outer portion 44 may be tapered to match the taper of the inner portion 14 of the shell 10 or the outer portion 44 may be cylindrical.

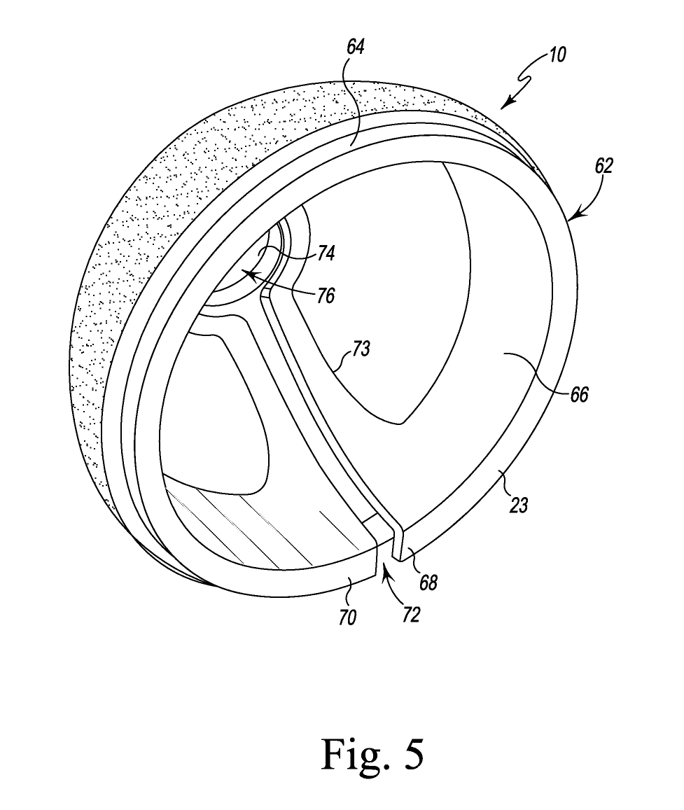

FIG. 5 illustrates yet another embodiment of a sleeve 62. The sleeve 62 has an outer portion 64 and an inner portion 66. The outer portion 64 engages the inner portion 14 of the shell 10. The outer portion 64 may be tapered to match the taper of the inner portion 14 of the shell 10 or the outer portion 64 may be cylindrical.

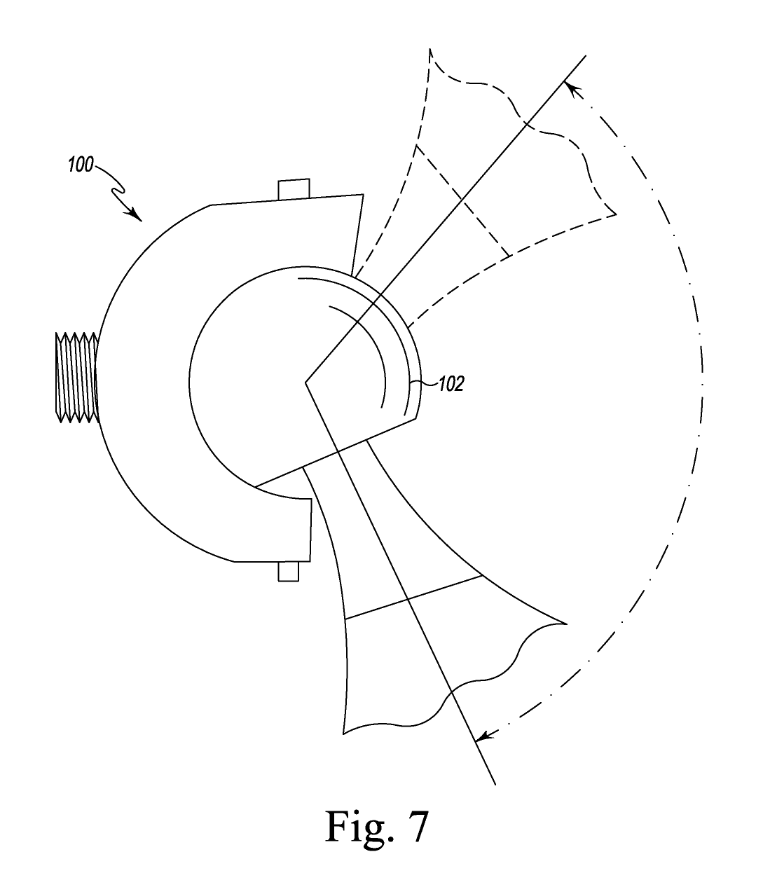

The sleeve 62 includes two ends, 68, 70 that create a recess 72. The two ends 68, 70 extend inwardly via an extension portion 73 toward an apex 74 of the shell 10. The two ends 68, 70 are uniform and encircle a hole 76 in the apex 74. The extension portion 73 can act as a spacer so the sleeve 62 may also work as a trial liner. During surgery, when the shell 10 is implanted, a user may sometimes put a trial liner 100 in the shell 10 (FIG. 7). The shell 10 and trial liner 100 then engage the head 102 (FIG. 7) to check range of motion (as illustrated by arrows in FIG. 7). In the embodiment illustrated in FIG. 5, the extension portion 73 allows the user to trial off of the sleeve 62 without the need for a separate trial liner. The extension portion 73 has the same thickness as a liner, so the range of motion of the head can be adequately tested. This reduces inventory and makes the process simpler for the user.

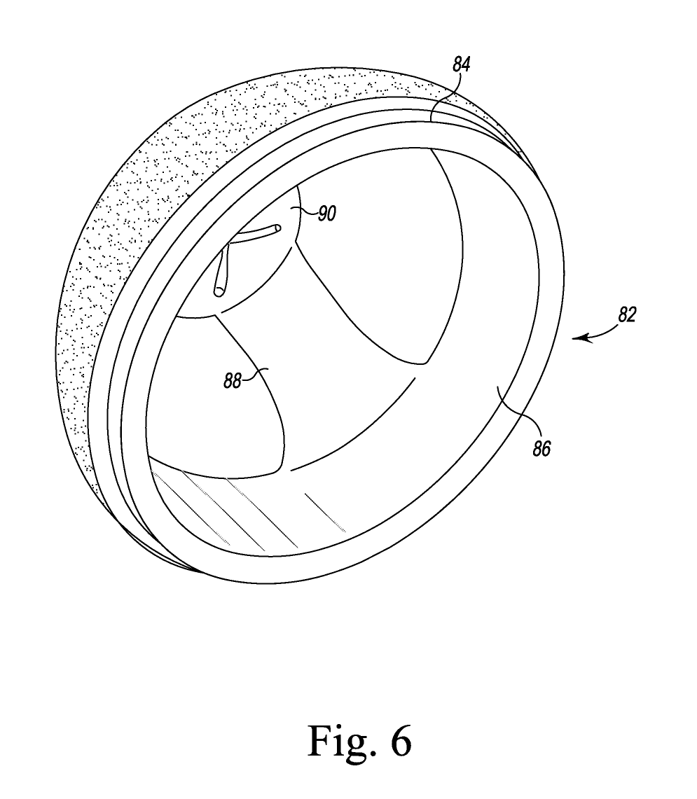

FIG. 6 shows another embodiment of a sleeve 82. The sleeve 82 includes an outer portion 84 and an inner portion 86. The outer portion 84 engages the inner portion 14 of the shell 10. The outer portion 84 may be tapered to match the taper of the inner portion 14 of the shell 10 or the outer portion 84 may be cylindrical. In this embodiment, the sleeve 82 is a single piece that includes an extension portion 88 that extends toward the apex of the shell 10. In this embodiment, the extension portion 88 ends in a cap 90 that extends over the apex. The extension portion 84 may also be used as a trial liner as described above.

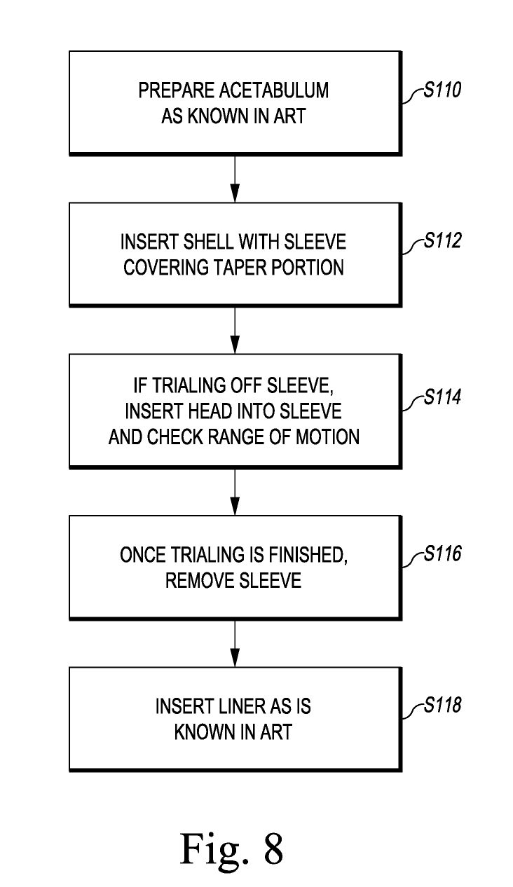

Turning now to FIG. 8, a method of how the sleeves as described above may be used. First, the acetabulum is prepared as is known in the art (step s110). This may be done by traditional reaming. Next, the shell 10 and the sleeve 22, 42, 62, or 82 are inserted into the prepared acetabulum (step s112). The sleeve 22, 42, 62, or 82 is covering the taper 16 of the shell 10. The user then may choose to trial off the shell 10 and sleeve 22, 42, 62, or 82 combination by inserting the head 100 and checking the range of motion at step s114. After the trial reduction is finished, the user removes the sleeve 22, 42, 62, or 82 at step s116, and the liner may be inserted (step s118). The user may choose to not trial off the sleeve 22, 42, 62, or 82 and may skip to step s116.

Although the present invention and its advantages have been described in detail, it should be understood that various changes, substitutions, and alterations can be made therein without departing from the spirit and scope of the present invention as defined by the appended claims.

1. An acetabular shell system comprising:
an acetabular shell having a convex outer portion and a concave inner portion; and
a polymer sleeve having an outer portion sized and shaped to engage at least a portion of the concave inner portion of the acetabular shell.

2. The shell system of claim 1, wherein the concave inner portion includes a locking mechanism and the polymer sleeve extends over the locking mechanism.

3. The shell system of claim 2, wherein the locking mechanism includes a female taper and the outer portion of the polymer sleeve includes a taper to engage the female taper.

4. The shell system of claim 2, wherein the locking mechanism includes a female taper and the outer portion of the polymer sleeve is cylindrical.

5. The shell system of claim 1, wherein, when the shell and sleeve are assembled, the sleeve including a rim which extends above of the inner portion of the shell.

6. The shell system of claim 1, wherein the sleeve includes a pull tab extending beyond the outer portion of the sleeve which engages the shell.

7. The shell system of claim 1, wherein the inner portion of the shell includes an apex and the sleeve includes an extension portion that extends toward the apex of the shell.

8. The shell system of claim 7, wherein the apex of the shell includes a screw hole and the extension portion of the sleeve extends around the screw hole.

9. The shell system of claim 7, wherein the extension portion of the sleeve includes a cap which extends over the apex of the shell.

10. A method for performing hip arthroplasty, the method comprising:
inserting an acetabular shell into a prepared acetabulum, the acetabular shell having a taper and a sleeve covering the taper;
inserting the head into the shell and sleeve;
testing the range of motion of the head in the shell and sleeve; and
removing the sleeve from the shell.

11. The method of claim 10, wherein the shell includes a locking portion and the sleeve extends over the locking portion.

12. The method of claim 10, wherein the sleeve includes an extension portion that extends towards an apex of the shell.

13. The method of claim 10, wherein the sleeve has the same thickness as a liner to be inserted into the shell.
